(19) United States
(12) Patent Application Publication
(10) Pub. No.: US 2023/0404702 A1
(43) Pub. Date: Dec. 21, 2023

(54) USE OF EXTERNAL CAMERAS IN ROBOTIC SURGICAL PROCEDURES

(71) Applicant: Asensus Surgical US, Inc., Durham, NC (US)

(72) Inventors: Kevin Andrew Hufford, Cary, NC (US); Nevo Yokev, Haifa (IL)

(21) Appl. No.: 18/092,194

(22) Filed: Dec. 30, 2022

Related U.S. Application Data
(60) Provisional application No. 63/295,258, filed on Dec. 30, 2021.
(60) Provisional application No. 63/295,380, filed on Dec. 30, 2021.
(60) Provisional application No. 63/295,167, filed on Dec. 30, 2021.
(60) Provisional application No. 63/295,185, filed on Dec. 30, 2021.

(51) Int. Cl. A61B 90/00

(52) U.S. Cl. CPC A61B 90/361

(57) ABSTRACT

One or more imagers are positioned in an operating room to capture images of equipment, instruments and or personnel in the operating room. A processor receives image data from the imagers and determines the relevant positions of the objects of interest. The user is with visual or auditory feedback, haptic guidance, or other feedback relative to the relevant positions to facilitate surgical setup, positioning of robotic manipulators, or to alert the user to close proximity of the manipulators.

This application claims the benefit of the following US Provisional Applications: U.S. 63/295,167, U.S. 63/295,380, U.S. 63/295,258, and U.S. 63/295,185, each filed Dec. 30, 2021, and each incorporated herein by reference.

### BACKGROUND

There are several types of surgical robotic systems on the market or under development. Some surgical robotic systems use a plurality of robotic arms. Each arm carries a surgical instrument, or the camera used to capture images from within the body for display on a monitor. Each of these types of robotic systems uses motors to position and/or orient the camera and instruments and to, where applicable, actuate the instruments. Typical configurations allow two or three instruments and the camera to be supported and manipulated by the system. Input to the system is generated based on input from a surgeon positioned at a surgeon console, typically using input devices such as input handles and a foot pedal. Motion and actuation of the surgical instruments and the camera is controlled based on the user input. The image captured by the camera is shown on a display at the surgeon console. The console may be located patient-side, within the sterile field, or outside of the sterile field.

In some surgical robot systems, the arms are mounted on one or more bases moveable along the floor in the surgical suite. For example, the Senhance Surgical System marketed by Asensus Surgical, Inc. uses a plurality of separate robotic arms, each carried on a separate base. In other systems, a first base might carry a first pair of arms, and a second base might carry one or more additional arms.

Knowledge of the relative positioning of equipment in the operating room can be important at various stages of the procedure. During setup of the operating room, the manipulators and other equipment such as the patent bed should be in ideal relative positions in order to optimize performance of the robotic surgical manipulators during the surgical procedure. Knowledge of the location of trocars positioned in incisions in the patient can be useful as instruments carried by the manipulator are inserted into the trocars, or (in some systems) as the end effector of the manipulator is docked to the trocar.

During the surgical procedure, the surgical team may need to move certain components of the surgical system. This may come about if the patient must be repositioned (e.g., by adjusting the position and or orientation of the operating table), or if the positions of other operating room equipment is changed. Moreover, awareness of the proximity between robotic manipulators and other manipulators, equipment or personnel in the operating room during the procedure beneficial for avoiding unintended contact or collisions. For surgical robotic systems having multiple arms that emanate from a common base, monitoring the relative position can be performed simply based on known kinematics. For surgical robotic systems in which the robotic arms are mounted on separate carts that may be individually moved, acquiring the relative positioning is more difficult. In some robotic surgical systems, a force-torque sensor and/or an IMU (inertial measurement unit)/accelerometer may be used to collect information from the surgical site as well as to detect collisions between the most distal portions of manipulators. However, it may be further desirable to predict or detect collisions between not only the most distal portions of the manipulator, but also more proximal portions that may be on the more proximal side of a distally positioned force-torque sensor.

This application describes systems and methods for using imagers/cameras in the operating room to aid in set-up of relevant equipment and personnel, and/or to facilitate performance of the surgical procedure.

This application further describes a system and method that uses cameras on the end effector of a manipulator to, among other things, gather information that aids the system in determining the relative position between the end effector or a corresponding instrument and a trocar, the patient, the patient table, etc. Use of these features can reduce the amount of personnel time and surgical suite time spent performing these tasks, and, therefore, reduce the procedure cost of the surgery.

Commonly owned US Publication No. US/2020/0205911, which is incorporated by reference, describes use of computer vision to determine the relative positions of manipulator bases within the operating room. As described in that application, one or more cameras are positioned to generate images of a portion of the operating room, including the robotic manipulators, or instruments carried by the robotic manipulators. Image processing is used to detect the robotic system components on the images captured by the camera. Once the components are detected in the image for each manipulator, the relative positions of the bases within the room may be determined. Commonly owned and co-pending Application Nos. 17/944,170, filed Sep. 13, 2022, which is incorporated herein by reference, describes use of externally positioned imagers to determine the relative positioning of manipulator arms, surgeon console, patient bed, etc. Commonly owned U.S. Ser. No. 18/091,292, filed Dec. 29, 2022, describes use of imagers on a robotic manipulator end effector to detect proximity between end effectors for purposes of collision avoidance. Concepts described in those applications are relevant to the present disclosure and may be combined with the features or steps disclosed in this application.

### BRIEF DESCRIPTION OF THE DRAWINGS

FIG. 1 is a perspective view of a robot-assisted surgical system on which the configurations described herein may be included;

FIG. 2 is a perspective view of a robotic manipulator arm with an instrument assembly mounted to the end effector;

FIG. 3 is a perspective view showing the end effector of the manipulator of FIG. 2, with the surgical instrument mounted to the end effector;

FIG. 4 is a perspective view similar to FIG. 3, showing the surgical instrument separated from the end effector;

FIG. 5 schematically shows a cross-section view of an end effector, taken transverse to the longitudinal axis of the end effector, utilizing an arrangement of detectors to detecting proximity of the end effector to other components or personnel;

FIG. 6 shows a plan view of two end effectors with mounted cameras, and schematically depicts the use of parabolic lenses to increase the fields of views of the cameras;

FIG. 7 is similar to FIG. 6 but shows an embodiment in which infrared LEDs are used to aid in proximity sensing.

FIG. 8 is a block diagram schematically depicting components of an exemplary proximity sensing system;

FIG. 9 schematically illustrates a series of steps for using the system depicted in FIG. 8.

FIG. 10 shows a top plan view of an operating room depicting a second embodiment which uses manipulator-mounted cameras to determine relative positions of subsystem components.

FIG. 11 is a front plan view of an exemplary camera suitable for use with the disclosed system and method;

FIG. 12 is a front elevation view of a manipulator of FIG. 1, with the camera system and microphone attached in accordance with a third embodiment;

FIG. 13 is a top plan view of the manipulator of FIG. 12;

FIG. 14 is a side elevation view of a surgeon console;

FIG. 15 is a block diagram schematically depicting the system of the third embodiment;

FIG. 16 is a perspective view of the surgeon console of FIG. 14 graphically depicting the 360 degree coverage area that may be captured using the camera arrangement shown in FIG. 14;

FIG. 17 is a plan view of the manipulators and a patient table graphically depicting the 300+ degree coverage areas that may be captured using the camera arrangements shown in FIGS. 12-14;

FIG. 18 schematically shows a side view of the end effector of FIG. 4 without the instrument attached, and further shows an external camera on the end effector in accordance with a fourth embodiment, as well as an adjacent patient, and a trocar positioned through an incision into the patient.

FIG. 19 is a schematic diagram illustrating components of the system of the fourth embodiment.

FIG. 20 is a schematic diagram illustrating components of the system of the fifth embodiment.

### DETAILED DESCRIPTION

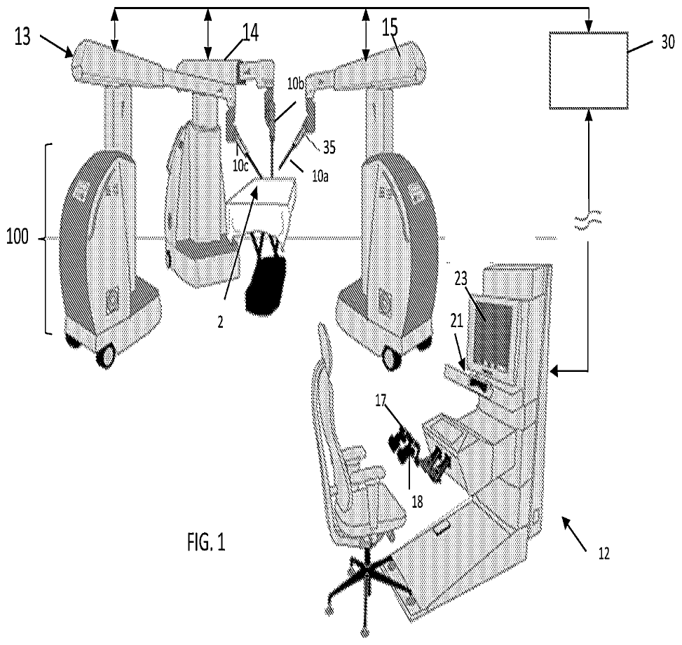

Although the inventions described herein may be used on a variety of robotic surgical systems, the embodiments will be described with reference to a system of the type shown in FIG. 1. In the illustrated system, a surgeon console 12 has two input devices such as handles 17, 18 that the surgeon selectively assigns to two of the robotic manipulators 13, 14, 15, allowing surgeon control of two of the surgical instruments 10a, 10b, and 10c disposed at the working site at any given time. To control a third one of the instruments disposed at the working site, one of the two handles 17, 18 may be operatively disengaged from one of the initial two instruments and then operatively paired with the third instrument. Or, as described below, an alternative form of input such as eye tracker 21 may generate user input for control of the third instrument. A fourth robotic manipulator, not shown in FIG. 1, may support and maneuver an additional instrument.

One of the instruments 10a, 10b, 10c is a laparoscopic camera that captures images for display on a display 23 at the surgeon console 12. The camera may be moved by its corresponding robotic manipulator using input from an eye tracker 21 or using input from one of the input devices 17, 18.

The input devices at the console may be equipped to provide the surgeon with tactile feedback so that the surgeon can feel on the input devices 17, 18 the forces exerted by the instruments on the patient's tissues.

A control unit 30 is operationally connected to the robotic arms and to the user interface. The control unit receives user input from the input devices corresponding to the desired movement of the surgical instruments, and the robotic arms are caused to manipulate the surgical instruments accordingly.

In this embodiment, each arm 13, 14, 15 is separately positionable within the operating room during surgical set up. In other words, the bases of the arms are independently moveable across the floor of the surgical room. These may be on any time of wheel, caster etc. that allow a user to easily change the position of the base on the floor of the operating room. This configuration differs from other systems that have multiple manipulator arms on a common base, and for which the relative positions of the arms can thus be kinematically determined by the system. However, although the inventive concepts described herein may be used in such systems if those systems are used together with other separately positionable components.

The patient bed 2 and the surgeon console 12, as well as other components such as the laparoscopic tower (not shown) may be likewise separately positionable.

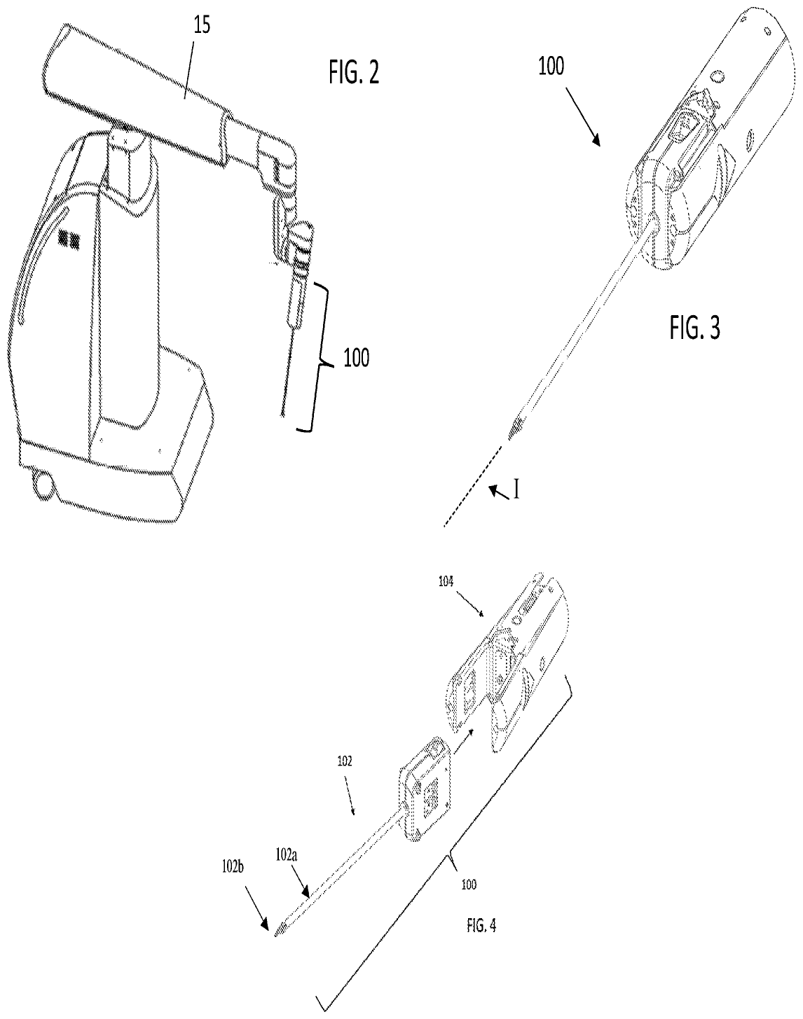

Referring to FIGS. 2-4, at the distal end of each manipulator 15 is an assembly 100 of a surgical instrument 102 and the manipulator's end effector 104. In FIGS. 3 and 4, the end effector 104 is shown separated from the manipulator for clarity, but in preferred embodiments the end effector is an integral component of the manipulator arm. The end effector 104 is configured to removably receive the instrument 102 as illustrated in FIG. 4. During a surgical procedure, the shaft 102a of the surgical instrument is positioned through an incision into a body cavity, so that the operative end 102b of the surgical instrument can be used for therapeutic and/or diagnostic purposes within the body cavity. The robotic manipulator robotically manipulates the instrument 102 in one or more degrees of freedom during a procedure. The movement preferably includes pivoting the instrument shaft 102a relative to the incision site (e.g., instrument pitch and/or yaw motion), and axially rotating the instrument about the longitudinal axis of the shaft. In some systems, this axial rotation of the instrument may be achieved by rotating the end effector 104 relative to the manipulator. Further details of the end effector may be found in commonly owned US Publication 2021/169595 entitled Compact Actuation Configuration and Expandable Instrument Receiver for Robotically Controlled Surgical Instruments, which is incorporated herein by reference. These figures show but one example of an end effector assembly 100 with which the disclosed system and method may be used, and it should be understood that the system and method are suitable for use with distinct types of end effectors.

### First Embodiment

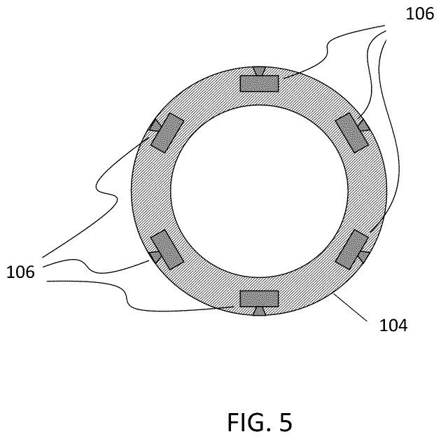

Referring to FIG. 5, a system for predicting collisions may include one or more imagers 106 (also referred to herein as cameras or detectors, etc.) positioned on a portion of a robotic manipulator, such as on the end effector 104. The view shown in FIG. 5 is a cross-section view of the end effector taken transverse to the longitudinal axis of the end effector (which typically will be parallel to the longitudinal axis of the instrument 102). The imagers are depicted as cameras positioned facing outwardly around the perimeter of the end effector as shown. In the drawing, the cameras are shown circumferentially positioned around the circumference of an end effector having a cylindrical cross-section, such that the lenses of the cameras are oriented radially outward from the end effector.

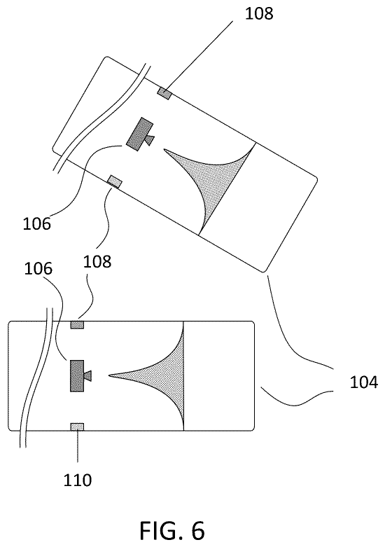

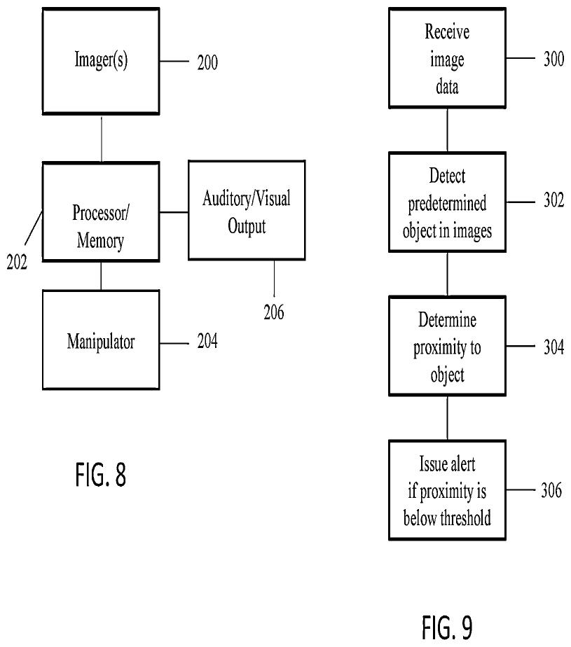

The imager system 200 is used in conjunction with at least one processor 202, as depicted in the block diagram shown in FIG. 8. The processor has a memory storing a computer program that includes instructions executable by the processor. These instructions, schematically represented in FIG. 9, including instructions to receive the image data corresponding to images captured the imager(s)/camera(s) (300), to execute an algorithm to detect equipment, personnel or other objects in the images (302), and to determine the distance between the manipulator and nearby equipment/personnel (the “proximal object”) or, at minimum, to determine that an object is in proximity to the end effector (304). The proximity detection step may rely on a variety of functions, including, for example, proximity detection, range estimation based on based on motion of feature(s) detected between frames of the captured image data, optical flow, three-dimensional distance determination based on image data from stereo cameras. Where multiple imagers are used, as in FIG. 5, image data from all or a plurality of the imagers may be used in the proximity detection step. In some embodiments, information from multiple cameras may be stitched together to acquire a seamless panoramic view/model that can be used to provide the system with situational awareness view with respect to each degree of freedom of movement of the end effector. In some embodiments, kinematic data from the robotic manipulator 204 may additionally be used to determine proximity, informing the processor where the relevant imagers of the end effector are relative to some fixed point on the corresponding manipulator or some other point in the operating room. Where markers are used on end effectors or other components of a robotic manipulator as discussed with respect to FIG. 6, kinematic data from the manipulator on which the LEDs or other markers are positioned may additionally be used by the proximity detection algorithm and/or by a collision avoidance algorithm.

In some embodiments, the algorithm further determines whether the distance is below a predetermined proximity threshold, and optionally takes an action if the distance is below the predetermined proximity threshold. Exemplary actions include generating an auditory alert or a visual alert (306). A visual alert might result in illumination of a light or LED, or in the display of an alert on a screen or monitor positioned. In either case, the device displaying or sounding the alert may be one on the manipulator, at the surgeon console, or elsewhere in the operating room. Other actions might include delivering a haptic alert to one or both of the surgeon controls 17, 18. For example, motors of the surgeon controls may be commanded to cause a vibration that will be felt by the surgeon holding the handles of the controls. Alternatively, the motors may be caused to increase resistance to further movement of the relevant control 17, 18 in a direction that would result in movement of the manipulator closer to the proximal object. Another action, which may be in addition to the alert 206 or an alternative to the alert 306, may be to terminate motion of the manipulator, or to terminate or slow-down motion of the manipulator that would result in movement of the manipulator closer to the proximal object. Similar actions may be taken in a simpler configuration where the sensitivity of the imagers/detectors is such that the system simply determines that there is an object in proximity to the end effector.

More complex actions may include providing updated motion to the manipulator or setup linkages with redundant kinematics to gradually move joints to minimize the likelihood of collisions between specific portions of the manipulator or to move the entire manipulator to overall configurations that are less likely to collide. This configuration optimization would occur in a move that is largely transparent to the user or could be a mode that the user enables when it is determined to be safe to do so. Safe contexts for use of the feature might include times when there are no surgical assistants working near the manipulator, when the instruments are in the trocars or not yet installed on the end effector.

In some implementations, the collision prediction/detection algorithms are processed for a single arm only on its own processing unit. In other implementations, they are processed in a single, central processing unit that collects information from a variety of inputs/manipulators/systems and then provides input commands to arms or other system components.

In a modified embodiment, imagers on the end effector might include one or more camera(s) having a parabolic lens, an axisymmetric lens or a reflector. Such lenses and reflectors allow a single lens to cover a very wide field of view. In configurations using them, the processor 202 is further programmed to mathematical unwarp the images captured by the image data into an appropriate spatial relationship. Some implementations may be configured to additionally permit forward viewing using the imager, such as by providing a gap or window in the parabolic lens, asymmetric lens or reflector. The shape(s) of the reflectors chosen for this embodiment may be selected to allow for targeting viewing of regions of interest, such as regions where problematic proximal objects are most likely to be found. Other implementations may use two cameras, one to cover each hemisphere and allow for use of the central axis of the structure for other purposes.

In alternative embodiments, omni-directional cameras may be used for sensing proximity between end effectors or other components. One or more such omni-directional cameras may be positioned on the end effector, elsewhere on the manipulator arm (e.g., high on the vertical column of the arm shown in FIG. 2, or on the horizontally extending boom), or at a high point in the operating room, such as on a ceiling fixture, cart, laparoscopic tower, etc.

As shown in FIG. 6, end effectors (or other potential proximal objects) in any of the disclosed embodiments may include known features, patterns, fiducials, LEDs that may be detected in the image data captured by the cameras and used for predicting potential collisions. The LEDs may vary in color depending on their position on the end effector, allowing the system to determine through image analysis which end effector or other proximal object is being captured by the relevant imagers. For example, for each end effector shown in FIG. 6, a green LED 110 is positioned on the right side of the end effector and a red LED 108 is positioned on the left side.

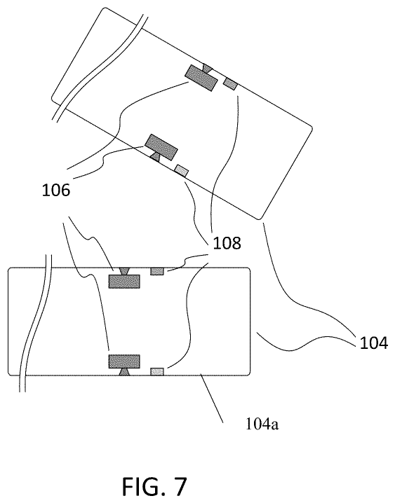

Infrared (IR) LEDs may be used in some embodiments for tracking and collision detection, as illustrated in FIG. 7. For example, LEDs that emit infrared wavelengths of light may be installed on the end effector or other elements of the robotic surgical system. Infrared light may transmit through sterile drape material so that when the end effector is covered by a sterile drape for surgery, the infrared light will transmit through it and can thus be detected by the imagers of the other end effectors. In some embodiments, the IR LEDs may be positioned beneath the housing/skin 104a (FIG. 7) enclosing the internal components of the end effector, since the IR light can transmit through visibly opaque materials. These LEDs may be single, or may be arranged in a certain pattern, and/or may use flash/blink patterns to provide different information, or to differentiate between elements and/or sides of a robot part. These LEDs or patterns of LEDs may be detected with an optical detector or a camera. While IR LEDs may be preferable, LEDs that emit in alternate or additional wavelengths (visible or invisible, RGB, etc.) are within the scope of the invention. Techniques described co-pending application Ser. No. 17/944,170 may be used to determine the distances from the optical detector or camera to the tracked component.

As described elsewhere in this application, although these embodiments are described with respect to the end effector of a manipulator, the same principles may be used to obtain overall situational awareness in the OR, potentially with a similar camera/lens/reflector configuration mounted on another portion of a manipulator arm, the vertical axis of the manipulator arm, etc.

### Second Embodiment

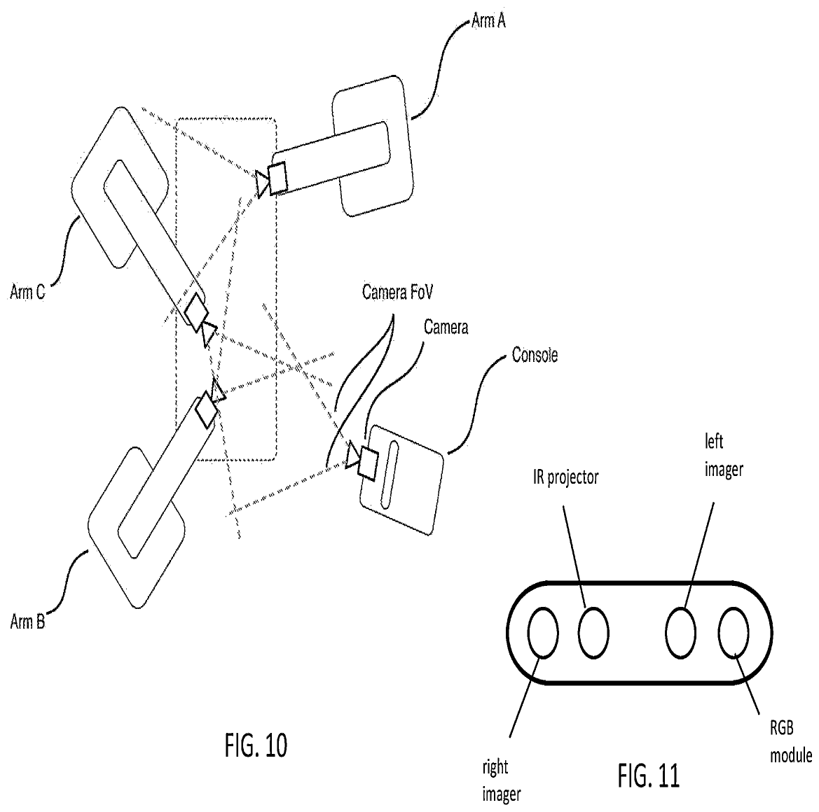

An alternative embodiment will next be described with respect to FIGS. 10 and 11. In this embodiment, the system includes an imager or series of imagers used to provide information about relative positioning between the manipulator arms, between manipulator arms and the patient and/or bedside staff, between manipulator arms and the operating table or other equipment. As with the previously-discussed embodiment, the imagers are external imagers (also referred to as cameras), i.e., cameras other than the cameras used to provide a view of the surgical workspace within the patient anatomy. The camera(s) may be mounted to or installed in one or more components of a surgical robotic system, including on one or more of the manipulators and/or the surgeon console as shown in FIG. 10, or a portion of the patient bed, etc. The camera(s) may alternatively or additionally be mounted to one or more of a variety of other structures, including, but not limited to, in or onto the ceiling booms, in or onto the ceiling, in the light fixtures, on walls, laparoscopic towers, racks, movable bases, or poles. They may be integrated in the manipulator arms or other operating room equipment or structures, or they may be removable.

These cameras may be of multiple types, including RGB-D, stereo, monocular, etc., or a combination thereof. RGB-D Cameras provide not only a traditional red-green-blue camera image, but also provide co-located depth information that provides or may be used to infer 3-dimensional data about the scene. An example of these may be the Intel Real Sense series of cameras in which a stereo pair of imagers is paired with an IR pattern projector, as well as an RGB imager. See FIG. 11. These external cameras provide the advantage of being able to provide information about a large portion of a robotic manipulator, not just where particular sensors on the manipulator are located.

Communication between the cameras and the surgical robotic system may vary, and may include both wired and wireless connections, and includes IP-network-based communication as well as more direct communication types and protocols that are used to communicate imager-based or sensor data.

This information may be provided to an individual manipulator arm and its collision avoidance system, or into an overall system responsible for providing collision avoidance. More particularly, the system analyzes the camera images to determine relative spatial relationships between items in an operating room, so that when distances between those items fall below a predetermined distance, the system may intervene to prevent collisions or minimize damage from collisions. Cameras may also be used to identify any or all of: relative robot base positions, relative positioning between elements of surgical robotic system, patient positioning, and trocar positioning.

In some implementations, this camera or series of cameras may also be used to detect motion of the OR table (or bed) and allow for an appropriate system response. This may be done by tracking the overall shape created by the patient, drape, and table, tracking of the exposed surgical site (skin), the trocar(s), or any combination thereof.

This system response may be used to move the manipulator arms in coordination with the OR table movement to prevent injury to the patient and improve OR efficiency. This external camera data may be combined with other sensors, such as data received from force-torque sensors mounted on the robotic manipulators. This information may be used for a variety of purposes, such as to update the remote-center-of-motion (fulcrum) of the robotic manipulator.

If a boom light, laparoscopic tower, or other equipment is moved within the reach of a manipulator, it is important for the system to recognize that a previously vacant space is now occupied and adjust accordingly to avoid collisions.

Use of the determined proximity information and the prediction of the potential for collision may be similar to that described with respect to the earlier embodiments. The information derived from the camera data provided may be of a variety of types, some with increasing richness of information. Resulting methods may include, but are not limited to:

Alerts provided to the user. Where intelligent cameras are used, they may provide automatic alerting if a boundary around a system component is about to be violated

Bounding boxes created around each element of interest in the camera(s) field of view (via computer vision). An alert is provided if these bounding boxes are about to cross (or within a definable margin or safe zone).

The one or more processors may generate and store an overall 3D model created from data collected by the camera or series of cameras. The processor may then infer or determine relative positions of the equipment based on the 3D model, predict an impending collision and issue an alert and/or take intervening action to prevent the collision.

The disclosed system provides a number of advantages, including, the ability able to detect a potential collision before it occurs, the ability to monitor moving equipment/personnel throughout the entire operating room, or a large portion of it, and the ability to generate real-time information about a large portion of a robotic manipulator, not just where sensors are located. In contrast, in some robotic systems having force-torque sensors only on the distal portion of a robotic manipulator, only collisions distal to the sensor can be detected but those sensors.)

### Third Embodiment

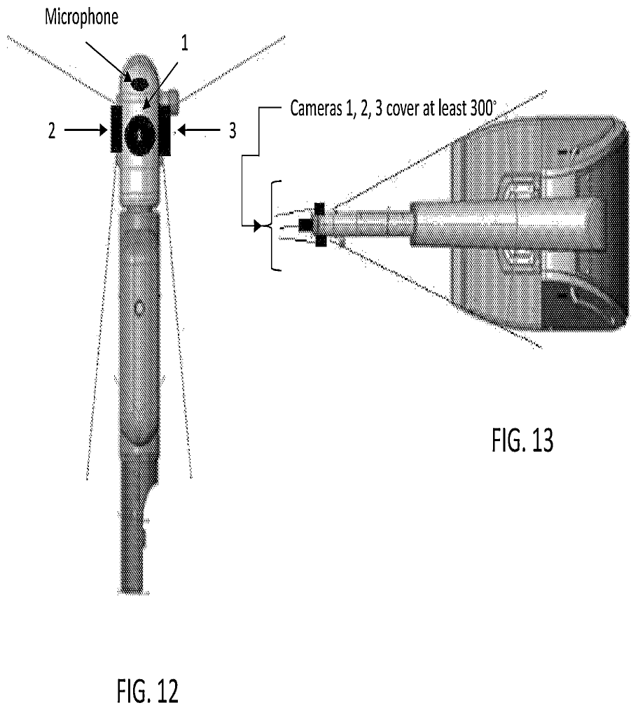

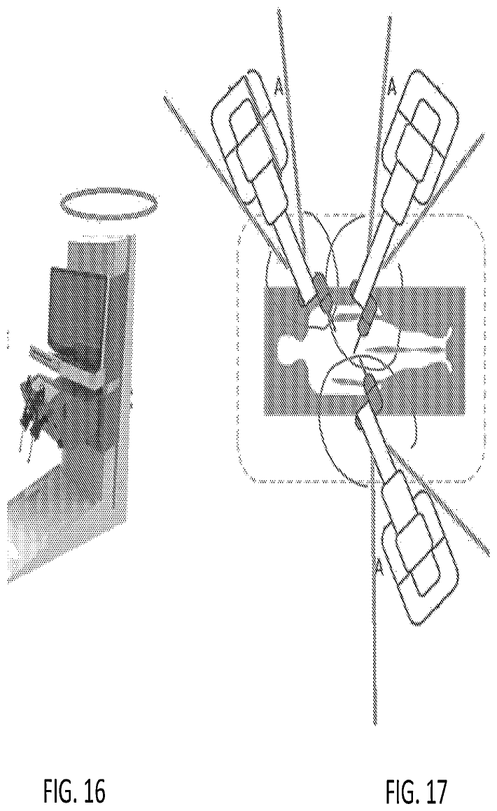

In a third embodiment, shown in FIGS. 12-17, an alternative configuration of external imagers is shown. These figures show the imagers in combination with optional microphone(s) as part of a real-time operating room sensor system.

Referring to FIG. 12, one or more of the manipulators includes a plurality of cameras. In the embodiment shown, the cameras comprise a system of three cameras that collectively provide a 300 degree or larger view of the surrounding area. This view is depicted by the arc draw around each arm in the view of FIG. 17. In the configuration of cameras shown in FIGS. 12 and 5, a first camera 1 faces forward, while a second camera 2 faces right and a third camera 3 faces left (180 degrees from camera 2 and 90 degrees from camera 1). The camera orientations may differ from what is described here without departing from the scope of the invention. In addition, a microphone is positioned on the manipulator as shown.

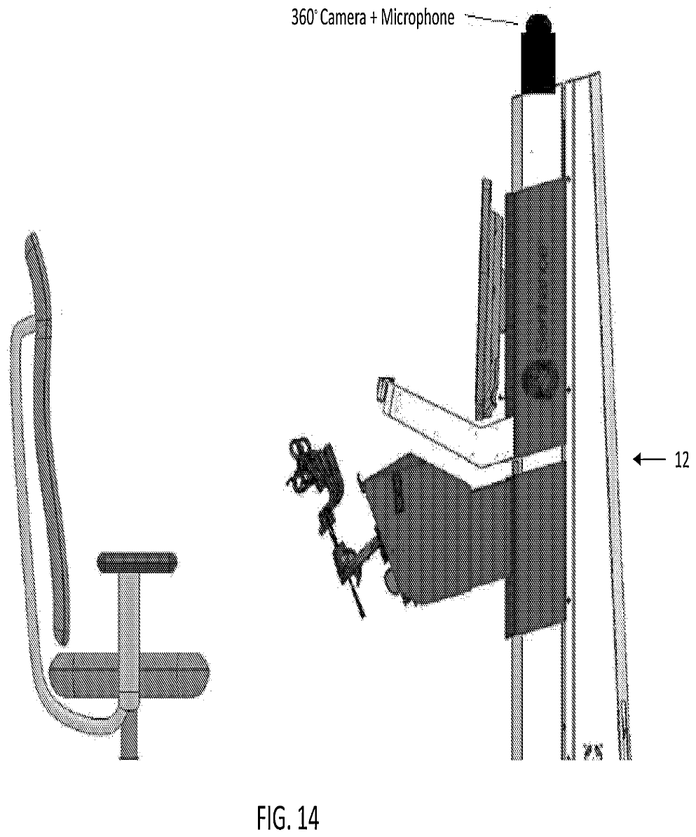

The surgeon console may also be optionally equipped with a camera and microphone as depicted in FIG. 14. The camera may be one that can capture a 360 view of the operating room.

The sensor system allows various components of the surgical system (e.g., manipulators, cockpit, laparoscopic tower, patient table etc.) in addition to other OR equipment and staff to be monitored in real time. This allows alerts to be generated or actions to be taken to avoid collisions, and can help guide the surgical team in optimal placement of the manipulators for each procedure etc.

The sensor system may also detect operating room alerts and insure they have been recognized by the surgeon. For example, the cameras and/or microphone may detect flashing lights, auditory alerts, screen data etc. from components in the operating room, and in response generate feedback to the surgeon (and/or the surgeon team) further drawing attention to them in case they were not originally noticed. For example, auditory data received by the microphones may be identified as indicating a certain change in operating mode, an error status, or the like, and generate a text or other visual alert displayed on the surgeon console or other displays within the operating room. Similarly, certain light patterns or blinking patterns of lights on the manipulators or other equipment in the room may be recognized and characterized using text or graphics on the surgeon console display or another display.

The microphone and camera systems may further allow the system to collect usage data for the robotic system. Such information can be used to allow analysis of the daily base use of the system in order to facilitate improvements in system set-up and use, ultimately allowing increases in efficiency. Data collected may also be used to generate records of the surgical procedure for use in patient records or training. For example, one or more of the microphones can capture voice notes or descriptions of the surgery. The surgeon or other operating room personnel can describe what they are doing, or their instructions to a resident or other team member may be recorded and then captured as text and synchronized with other the logged data. The surgeon can describe the current stage in the surgical procedure, an action s/he is taking, what organs are visible in the scene, problems that s/he sees and so on. In some embodiments this feature may be combined with concepts described in co-pending related application Ser. No. 17/495,792, Surgical Record Creation Using Computer Recognition of Surgical Events, which is incorporated herein by reference.

The microphones may be configured to permit the users to deliver voice commands to direct certain functions of the surgical system.

Finally, the microphones and cameras may be used in conjunction with audio output devices such as speakers or headsets to facilitate tele-collaboration as described in co-pending Application Ser. No. 17/460,128, entitled Tele-Collaboration During Robotic Surgical Procedures, which is incorporated herein by reference.

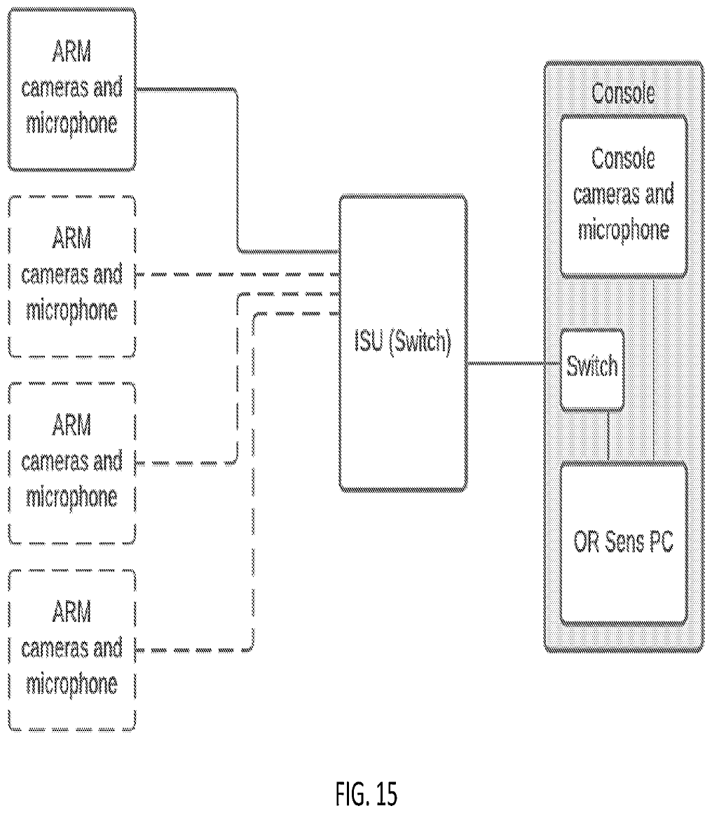

The camera system is used in conjunction with at least one processor, identified in FIG. 15 as the “ISU” and the “OR Sens PC”. The processor has a memory storing a computer program that includes instructions executable by the processor to receive image data captured by the camera(s) and auditory data captured by the microphones, and to execute an algorithm to conduct the functions described in this application and/or the applications attached at the Appendix.

### Fourth Embodiment

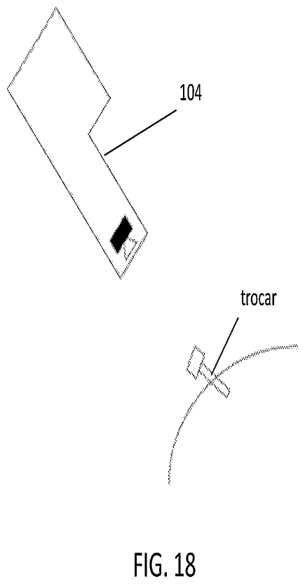

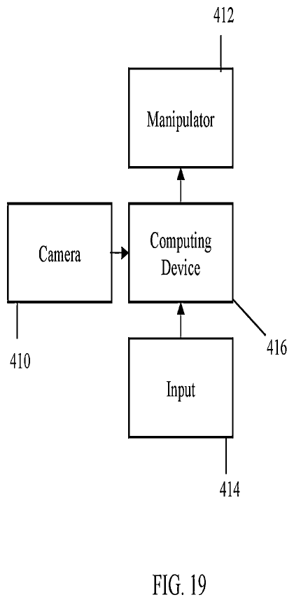

A system and method according to a fourth embodiment uses cameras on the end effector of a manipulator to, among other things, gather information that aids the system in determining the relative position between the end effector or a corresponding instrument and a trocar, the patient, the patient table, etc. The system includes a camera or series of cameras at a distal portion of a manipulator, such as on the end effector as shown in FIG. 18. As with the prior embodiments, the camera is an external camera (i.e., one other than the endoscopic/laparoscopic camera used to provide a view of the surgical workspace within the patient anatomy).

These cameras may be of multiple types, including RGB-D, stereo, one or more monocular cameras, etc., or a combination thereof. RGB-D Cameras provide not only a traditional red-green-blue camera image, but also provide co-located depth information that provides or may be used to infer 3-dimensional data about the scene. An example of these may be the Intel RealSense series of cameras in which a stereo pair of imagers is paired with an IR pattern projector, as well as an RGB imager. See FIG. 11.

Data from the camera may be used for a variety of functions. In one embodiment, the camera is positioned on the end effector so that it is forward-looking towards the incision site during use. With this configuration, the camera may be used to facilitate instrument insertion into the incision, and or docking of the end effector to the proximal end of the instrument (or, in alternative embodiment, to the trocar). In a typical robotic surgical procedure, surgical staff forms an incision in the patient, and inserts a trocar through the incision. The purpose of the trocar is to provide a conduit through which the instrument is inserted. Once the trocar is positioned, the instrument tip is guided through the trocar in one of several ways. A first option is performed with the instrument already mounted to the end effector. In this case, the end effector which supports the instrument is hand-guided by the user until the instrument is aligned with the lumen of the trocar. Once aligned, the instrument is inserted through the trocar so that its tip is within the patient's body cavity. When this method is used, data from the camera can be used by the system to recognize the trocar and the orientation of the longitudinal axis of its lumen. Once the axis is determined, the system can first allow hand-guiding movements in directions that will guide the instrument so the insertion axis I (FIG. 3) of the instrument aligns with the longitudinal axis of the trocar. Once the axes are aligned, the system can limit the hand-guiding function of the manipulator so the instrument axis can only be moved along the longitudinal axis of the trocar. The “limits” may be hard limits that prevent any deviation from the desired movements, or they can be “soft” limits that give haptic constraints when the hand-guiding movements deviate from those that are optimal. Such soft haptic constraints are intended to gently guide the user to insert only along the trocar axis. As the instrument is inserted more fully through the trocar, the axis of the trocar may change. Some embodiments of the system continue monitoring the trocar using the camera and will adjust the haptic guidance accordingly to ensure continued alignment of the instrument axis and trocar axis.

A second option involves first inserting the surgical instrument through the trocar, and then moving the end effector towards the surgical instrument so the two can be engaged (e.g., as shown in FIG. 4). When this method is used, data from the camera can be used by the system to recognize the trocar and the orientation of the longitudinal axis of its lumen as described above. Once the axis is determined, the system can first allow hand-guiding movements in directions that will guide the end effector such that the insertion axis I of the instrument (which, even though the instrument is not presently docket, is known to the system relative to the geometry of the end effector) will align with the longitudinal axis of the trocar once the instrument and end effector are docked. Once the axes are aligned, the system can limit the hand-guiding function of the manipulator so that the end effector can only be moved such that the insertion axis will move along the longitudinal axis of the trocar. In an alternative embodiment, the data from the camera will be used to guide the user to align the end effector with a portion of the instrument detected by the camera.

Certain embodiments might use data from the camera to facilitate semi-autonomous movement of the end effector into alignment with the trocar. This automatic movement may require contemporaneous action by the user. For example, the system might have limits requiring a user to be pressing a button or contacting a portion of the manipulator (i.e., as a “dead-man switch”) for movement of the end effector to occur. When activated, the system may be configured to command the motors of the manipulator to cause the end effector to gently “float” into alignment with the trocar, as if a rubber band or spring is pulling it into alignment.

The data from the camera may allow the system to recognize one or more characteristics of the trocar if it is necessary to identify and differentiate between trocars. For example, the system may be configured to recognize the trocar's orifice (size, shape, position relative to other detectable features), exterior shape, shaft, markings, text, logos etc.

In a modified embodiment, the system may further be configured so that data from the camera is used to recognize or determine the type of instrument (tip type, length etc.) that the end effector is attached to or adjacent to for instrument docking (e.g., as shown in FIG. 4). Once the instrument type is determined, hand-guiding limits towards the trocar may be further tailored to prevent the end effector from being advanced by a distance that would exceed the optimal insertion depth for the instrument. Alternatively, or additionally, the recognized instrument type information may be used by the surgical system for controlling movement of the manipulator during surgery in a manner that is appropriate for the type and/or geometry of the instrument. As another alternative, the recognized instrument type information may be used in creating digital records or charts that include records of which instruments were used at certain points in the procedure. Instrument types may be recognized from camera data in many ways, including based on shapes, markings or other features.

Other uses for data obtained by the cameras includes monitoring proximity between end effectors of different manipulators for purposes of collision avoidance, as described with respect to the prior embodiments and in the co-pending applications referenced above.

The cameras may also recognize user hand gestures, allowing personnel to change a manipulator's position or orientation using a hand gesture instead of physically touching the manipulator. This can be particularly useful to allow a non-sterile user to reposition a manipulator.

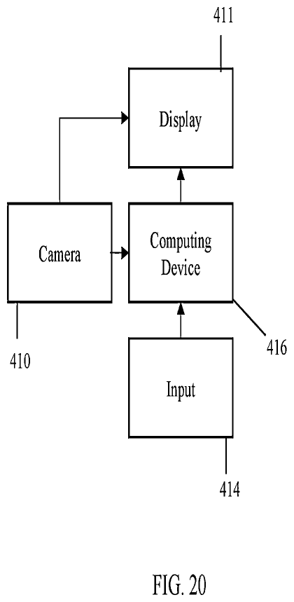

FIG. 19 schematically illustrates a system according to the disclosed concepts. The system includes at least one camera 410, which is preferably distally mounted on a manipulator 412 as described. A user input 414 may optionally be included to allow a user to give input to the system that is pertinent to the surgical set-up. The input may indicate the type of surgery, patient data such as body mass index (BMI), the lengths of instruments to be mounted to the surgical system, the viewing angle of the endoscope to be used for the procedure. At least one processor 416 is configured to perform the functions described above, including all or a subset of the following steps:
receive procedure-related user input;
receive image data from the camera(s);
determine characteristics of the trocar, such as the position and orientation of the trocar's lumen relative to the end effector and/or instrument shaft based on the image data;
cause the manipulator's brakes and/or motors to create haptic limits or soft restraints such that, as the user hand guides the end effector towards the trocar, the haptic limits or soft restraints guide the end effector such that its insertion axis aligns with the longitudinal axis of the trocar.

The processor may further, optionally, determine a type or characteristics of the surgical instrument, and set hand guiding restraints/limits or control manipulator movement in accordance with type, length, geometry or other characteristics of the surgical instrument.

Note that the one or more processors may be processors integral with the camera or separate components that receive signals from the camera.

### Fifth Embodiment

A system and method according to a fifth embodiment includes a camera or series of cameras used to provide information about relative positioning between the manipulator arms, between manipulator arms and the patient and/or bedside staff, between manipulator arms and the operating table or other equipment. As described for previous embodiments and depicted in FIG. 10, the cameras are external cameras may be mounted to or installed in one or more components of a surgical robotic system, including on one or more of the manipulators and/or the surgeon console (e.g., as shown in FIG. 14), or a portion of the patient bed, etc. The camera(s) may alternatively or additionally be mounted to one or more of a variety of other structures, including, but not limited to, in or onto the ceiling booms, in or onto the ceiling, in the light fixtures, on walls, laparoscopic towers, racks, movable bases, or poles. They may be integrated in the manipulator arms or other operating room equipment or structures, or they may be removable.

These cameras may be of multiple types, including RGB-D, stereo, monocular, etc., or a combination thereof. RGB-D Cameras provide not only a traditional red-green-blue camera image, but also provide co-located depth information that provides or may be used to infer 3-dimensional data about the scene. An example of these may be the Intel RealSense series of cameras in which a stereo pair of imagers is paired with an IR pattern projector, as well as an RGB imager. See FIG. 11. These external cameras provide the advantage of being able to provide information about a large portion of a robotic manipulator, not just where particular sensors on the manipulator are located.

Information from these cameras is used to improve system setup prior to and/or during a surgical procedure. In some embodiments, the system generates feedback to the user based on the data from the camera. The feedback may be visual feedback that displays target positions for the manipulators, console, patient table or other moveable operating room equipment or personnel. A graphical display may provide outlines or graphics of optimal robotic manipulator base (or other equipment) placement, with the current location of the robotic manipulator bases also displayed, providing real-time feedback to the user as they wheel the bases into the correct positions and orientations. The display may change, or other visual or optical feedback may be given as the correct positions are achieved.

The visual feedback may be generated in the form of graphics on visual displays, or visual overlays on images of the operating room captured by the camera and displayed to the user. The display may be at the surgeon console. In other embodiments, the display may be on a device such as a tablet or smart phone, with the device's touch screen, microphone and/or one or more other devices (e.g., keyboard, mouse, stylus) used as the user input device 414. The display might instead be part of a heads up display or augmented reality headset. In some embodiments, optical tracking or other forms of tracking may be used in conjunction with those devices to allow the position of the display relative to the equipment to be known to or determined by the processor.

In some embodiments, the visual feedback is displayed as overlays on a display of images of the surgical site to assist the operating room staff in setting up for a robotic surgery case. More specifically, augmented reality is used to project an overlay, which may be a 3D overlay, of an optimized system setup for the robotic surgery case over the real-time display of the robotic arms (or on a transparent heads-up display the user can see through to see the manipulators and other components). This graphical display may provide outlines of optimal robotic manipulator base placement, with the current location of the robotic manipulator bases also displayed or visible through the transparent display, providing real-time feedback to the users as they wheel the bases into the correct positions and orientations. The display may change, or other visual or optical feedback may be given as the correct positions are achieved. Operating room staff reference the overlays while positioning the robotic arms in order to accurately position the robotic system in place to prepare for the operation.

In other embodiments, light projectors positioned in the operating room (e.g., using ceiling-mounted or boom-mounted lights) may project the target positions onto the floor or walls in order to show the user where the equipment should be positioned. The floor of the operating room may itself have a display on its surface that displays the optimal positions for the various movable pieces of equipment and personnel.

The optimal placement of manipulator bases and other equipment may be different for different intended surgical procedures. In some embodiments, optimized setup locations for the surgical system's arms may be established for different surgical procedures. These positions ensure the arm does not enter limited motion and prevents arm collision during surgery. The optimal placement for a given procedure may be sourced from a database of known procedures accessible by the processor, or may be determined automatically based on user input of trocar locations or based on automatic recognition of trocar locations via the referenced cameras or other sources.

Auditory cues may also or alternatively be given to guide the user's placement of the manipulator bases or other equipment to target positions.

During surgery, data received from the cameras can be used to provide intra-operative recommendations to reposition equipment in a manner similar to that performed during set-up.

In other systems, the system generates commands that result in automatic motion of robotic manipulator bases to target position.

Referring to FIG. 20, a system for providing feedback to guide pre- or intra-operative positioning of components of a surgical system or operating room personnel includes at least one camera 410 positionable to capture images of moveable equipment at a medical procedure site, and to generate other data such as 3D depth data or data that may be used to estimate the distance between the cameras and associated equipment. An image display 411 may display the captured image. A user input 414 may be included to allow a user to give input to the system that is pertinent to the surgical set-up. The input may indicate the type of surgery, patient data such as body mass index (BMI) and height, the numbers and/or types of bedside personnel, the lengths of instruments to be mounted to the surgical system, the viewing angle of the endoscope to be used for the procedure. In some embodiment, the one or more processors may also receive input corresponding to bed parameters (e.g., height, Trendelenburg/reverse Trendelenburg angle), instrument parameters; in other embodiments this data may be determined by the one or more processors based on data received from the cameras. At least one processor 416 is configured to perform all or a subset of the following steps:
receive procedure-related user input;
receive image data from the camera(s);
determine relative positions of a first subject and a second subject captured by the image data, where the subjects are equipment such as manipulators, surgeon console, patient table, etc., and optionally operating room personnel;
display the image in real time on an image display;
determine, based on the procedure-related input, a target position of at least one of the first subject and the second subject within the medical procedure site, and
provide feedback guiding the user to a target position for the first or second subject.

Note that the one or more processors may be processors integral with the camera or separate components that receive signals from the camera.

In the fifth and other embodiments, depending on the type of cameras used, the information provided from the cameras/sensors may be of a variety of types, some with increasing richness of information. Resulting methods may include, but are not limited to:
Intelligent cameras may be able to automatically create bounding boxes around elements in the operating room (OR) and provide location information to a processing/control unit.
Intelligent cameras may be able to recognize trocars (logos, shapes, etc.) and provide location information to a processing/control unit, as discussed in connection with the fourth embodiment.
A computer vision algorithm in the one or more processors unit may process the image to detect elements (the subject equipment or markers on the equipment) and infer positions of the equipment, etc.
The one or more processors may generate and store an overall 3D model created from data collected by the camera or series of cameras. The processor may then infer or determine relative positions of the equipment based on the 3D model.
Techniques described co-pending application Ser. No. 17/944,170, filed Sep. 13, 2022, which is incorporated by reference, may be used to determine the distances from the optical detector or camera to the tracked component. As described, LEDs or other fiducials may be used to aid in detection or recognition of the subject equipment.

The system and method allow the user to quickly and accurately setup a robotic surgical system having discrete, independently moveable components without the need for physically measuring distances between those devices. It may also be used in conjunction with manipulator set up procedures in accordance with U.S. Ser. No. 17/368,747, Augmented Reality Surgery Set-Up for Robotic Surgical Procedures, which is incorporated herein by reference.

The examples given above describe use of the system/method for positioning of robotic arms, but it should be appreciated that they may also be used to position other equipment as well as operating room personnel, including any of the following in any combination with the robotic arms: the patient, bedside staff, the patient, the OR table.

Any of the described embodiments may be modified to, in lieu of or in addition to providing feedback to the user to guide the user's placement of the robotic manipulators and other components/personnel, the system and method may be used for any of the following:
Initiating automatic motion of robotic manipulator bases to a desired position.
Displaying recommendations to the user about moving other elements (booms, laparoscopic column, etc.) within the operating room to alternate locations.
Initiating, or displaying recommendations to the user about, intra-operative adjustments or recommendations for adjustments to the surgical system

Concepts described in the co-pending and commonly owned applications referenced in this application are considered part of this disclosure and may be combined with the concepts described in this application.

For example, the methods and configurations used to determine the current relative positions of manipulator bases or other items of equipment described in those applications may be used in conjunction with the concepts described here. Additional, methods and configurations used to determine trocar positions may likewise be combined with concepts described in this application.

All prior patents and applications referenced herein are incorporated herein by reference.

We claim:

1. A robotic surgical system comprising:
a first robotic manipulator arm having a first base and a first end effector;
a second robotic manipulator arm having a second base and a second end effector;
each of the first base and the second base independently moveable on a floor of an operating room;
at least one camera positioned on at least one of the first robotic manipulator and the second robotic manipulator, the at least one camera positioned to capture images of the first manipulator and the second manipulator,
at least one processor having a memory, the memory storing an algorithm executable by the processor to detect movement of a portion of the first manipulator in proximity to a portion of the second manipulator.
